(19) United States
(12) Patent Application Publication
(10) Pub. No.: US 2007/0299025 A1
(43) Pub. Date: Dec. 27, 2007

(54) Method for Detecting the Risk of Cardiovascular Diseases Such as Acute Myocardial Infarction and Coronary Heart Disease By Analysing Defesin

(75) Inventors: Jukka T. Salonen, Kuopio (FI); Mia Pirskanen, Kuopio (FI); Jani Haarus, Kuopio (FI); Tomi-Pekka Tuomainen, Kuopio (FI); Faisel Yunus, Lahore (PK)

Correspondence Address: BIRCH STEWART KOLASCH & BIRCH, PO BOX 747, FALLS CHURCH, VA, 22040-0747, US

(73) Assignee: Oy Jurilab Ltd, Kuopio (FI)

(21) Appl. No.: 10/586,312

(22) PCT Filed: Jan. 17, 2005
(86) PCT No.: PCT/FI05/50007

§ 371 Date: Apr. 16, 2007

(30) Foreign Application Priority Data
Jan. 15, 2004 (FI) 20040052

(51) Int. Cl. A61K 31/7088; A01K 67/00; A61P 9/00; C07H 21/04; C12P 19/34; C12Q 1/00; C12Q 1/68; G01N 33/50

(52) U.S. Cl. 514/44; 435/40.5; 435/6; 436/94; 536/23.1; 536/24.31; 800/13

(57) ABSTRACT

The invention provides a method of identifying subject's susceptibility or predisposition to or risk of developing cardiovascular diseases such as myocardial infarction (MI) or coronary heart disease (CHD) by detecting gene polymorphisms and other gene mutations in a defensin gene from a biological sample of the subject and obtaining information concerning the family and medical history, blood, serum, plasma and urinary analytes of the subject. The invention also provides a multivariate model, a combination or algorithm of variables which best describes the probability of CHD, especially MI. The invention also relates to a test kit and software for accomplishing the method. The present invention also relates to a variant defensin gene.

This invention relates to a method to detect genetic variation in a defensin gene for the diagnosis of a risk of, or predisposition to, acute myocardial infarction (AMI) and coronary heart disease (CHD) in a subject, a method for targeting treatment in a subject, and a method for selecting subjects for studies testing anticoronary agents, as well as a method for the treatment and prevention of CHD and AMI. The present invention also provides a method of identifying subject's susceptibility to or risk of developing AMI or CHD by detecting gene polymorphisms from a biological sample of the subject and obtaining information concerning the family and medical history, serum or plasma analytes and clinical findings of the subject. The invention also provides a multivariate model, a combination or algorithm of variables which best describes the probability of AMI and CHD. The invention also relates to a test kit and software for accomplishing the method. Moreover, the invention relates to a nucleic acid influencing the production of a novel variant defensin protein as well as a method for screening a subject to determine if said subject is a carrier of variant gene that encodes said variant or non-variant defensin protein.

### FIELD OF THE INVENTION

The present invention is generally directed to a method for assessing the risk of CHD and AMI in an individual, such as a human. Specifically, the invention is directed to a method that utilises both genetic and phenotypic information as well as information obtained by questionnaires to construct a score that provides the probability of developing coronary heart disease. Furthermore, the invention provides a kit for carrying out the method. The kit can be used to set an etiology-based diagnosis of coronary heart disease and AMI for targeting of treatment and preventive interventions, such as dietary advice as well as stratification of the subject in clinical trials testing drugs and other interventions.

### BACKGROUND OF THE INVENTION

Coronary heart disease (CHD) is the major cause of death in the developed world The screening for conventional cardiovascular risk factors fails to identify more than 50% of the individuals who will present with acute coronary syndromes or AMI. Inflammation plays a role in both the development of atherosclerosis and the acute activation of the vascular wall with consequent local thrombosis and vasoconstriction. In many patients with unstable angina and AMI, systemic signs of inflammation are detectable. The use of systemic inflammatory markers, such as C-reactive protein as marker of disease activity and short- and long-term prognosis, seems to be of clinical value. Therefore, acute inflammatory reaction, detectable systematically, is a plausible risk factor for CHD and AMI.

As CHD is a polygenic disease, it is reasonable to assume that genetic variation in mechanisms important for the regulation of biochemical pathways that have a role in the development of atherosclerosis and CHD will be found to be associated with the pathogenesis and therapy of CHD.

One of the currently explored markers of inflammation is defensin. Defensins are a family of small cationic, antibiotic peptides that contain six cysteines in disulfide linkage. The peptides are abundant in phagocytes and small intestinal mucosa of humans and other mammals. They contribute to host defense against microbes and may participate in tissue inflammation and endocrine regulation during infection (Ganz and Lehrer 1995, Valore et al. 1998) and are a part of the innate immune system (Jia et al. 2001). There are two classes of defensin genes, α and β, that differ in their disulfide bond pairing, genomic organization, and in their tissue distributions. In addition to their broad spectrum antimicrobial properties, there is evidence that the β-defensins act as chemokines for immature dendritic cells and memory T cells, and thus may serve as an important bridge between the innate and adaptive immune systems (Jia et al. 2001, Hoover et al. 2001).

Defensins are normally sequestered in cytoplasmic granules with their primary site of action in phagolysosomes, although some peptide is released into the circulation during the course of infection or inflammation. Defensins have been found primarily in the intima of normal and atherosclerotic arteries, most prominently in association with intimal smooth muscle cells by immunohistochemisty. Defensins are also found in the media near the external elastic lamina and in some periadventitial vessels. This indicates the presence of defensins in the walls of human coronary arteries. The deposition of defensins in vessels may contribute to the pathophysiological consequences of inflammation in addition to their role in host defense (Barnathan et al. 1997).

Characteristically, the antimicrobial activity of the β-defensin peptides is salt sensitive and their killing is markedly reduced as the ionic strength of the solutions increases (i.e., NaCl>50 mM) (Schutte and McCray 2002).

The primary structure of each β-defensin gene product is characterized by small size, a six-cysteine motif, high cationic charge, and exquisite diversity beyond these features. The most characteristic feature of defensin proteins is their six-cysteine motif. Each β-defensin gene encodes a preproprotein that ranges in size from 59 to 80 amino acids with an average size of 65 amino acids. This gene product is then cleaved to create the mature peptide that ranges in size from 36 to 47 amino acids, with an average size of 45 amino acids (Schutte and McCray 2002) and molecular mass of 3-4 kD (Bensch at al. 1995).

At least 6 beta-defensins (HBD-1, HBD-2, HBD-3, HBD-4, HBD-5, HBD.6) have been characterized in humans. Human β-defensin-1 (HBD-1) was the first one to be characterized and isolated from the hemofiltrate of patients with end stage kidney disease undergoing dialysis (Lehmann et al. 2002). HBD-1 gene is expressed predominantly in urogenital epithelial organs such as kidney, urinary bladder, ureter and the female genital tract, with lesser expression in the pancreas, liver, and other epithelia Within We kidney, in situ hybridization indicates that HBD-1 is produced in distal tubules, loops of Henle, and collecting ducts. Human urine contains 10-100 μg/L of HBD-1 (Zucht et al. 1998, Ganz 2001).

The human β-defensin-1 (HBD-1) gene covers approximately 8 kB on chromosome 8p23·1 (Dork and Stuhrmann 1998) and is comprised of two exons separated by an intron that is usually 1.5 kb, but can be as large as 16 kb. The processed transcript varies from 300 to 400 nucleotide (nt) in length with a 5′ UTR 35 nt, an open reading frame of 200 nt, and a 3′ UTR of 100 nt. The first exon includes the 5′ UTR and encodes the leader domain of the preproprotein; the second exon encodes the mature peptide with the six-cysteine domain (Schutte and McCray 2002).

Thus, inflammatory mechanisms are important participants in the pathophysiology of CHD. The identification of useful markers of inflammation and host resistance (like defensins), of new therapeutic targets to interfere with these mechanisms, and the evaluation of the efficacy of anti-inflammatory treatments will allow progress in our ability to prevent and manage CHD and combat its complications.

### SUMMARY OF THE INVENTION

The object of this invention is to provide a method for screening a subject to assess if an individual is at risk to develop myocardial infarction or coronary heart disease, based on the genotype of a defensin gene and a method to target treatments and preventive therapies for CHD and AMI. The invention also provides methods for the treatment of CHD in a human or animal subject. A further object of the invention is to provide a method for the selection of experimental animals and human subjects for studies testing anticoronary and antihypertensive effects of drugs. Another object of the invention is a method for the selection of subjects for clinical trials testing anticoronary and antihypertensive drugs. A further object of the present invention is a method of identifying the risk of AMI and coronary heart disease by detecting gene polymorphisms from a biological sample of the subject. The information obtained from this method can be combined with other information concerning an individual, e.g. results from blood measurements, clinical examination and questionnaires. The genetic information includes data on mutations in genes associated with MI and/or coronary heart disease. The blood measurements include the determination of plasma or serum cholesterol and high-density lipoprotein cholesterol. The information to be collected by questionnaire includes information concerning gender, age, family and medical history such as the family history of CHD and diabetes. Clinical information collected by examination includes e.g. information concerning height, weight, hip and waist circumference, systolic and diastolic blood pressure, and heart rate.

More particularly, the invention provides a method for detecting genetic variation or polymorphism, i.e. a mutation, in a defensin gene comprising the steps of:
i) providing a biological sample taken from a subject to be tested,
ii) detecting the presence or absence of a variant genotype of the defensin gene in the biological sample, the presence of a variant defensin genotype indicating an increased risk of cardiovascular disease, such as CHD and AMI, in said subject.

Said defensin gene can be selected from the group consisting of: beta-defensin-1, beta-defensin-129, and alfa-defensin-5.

Moreover, the pattern of gene alleles can be further determined from the genes selected from the group consisting of:
a) alpha-_{2B}-adrenoceptor,
b) apolipoprotein B, and
c) beta-2-adrenergic receptor
   in order to confirm the risk of cardiovascular disease in said subject.

### DETAILED DESCRIPTION OF THE PREFERRED EMBODIMENTS OF THE INVENTION

In a preferred embodiment the invention comprises the assessment of genetic variants in a defensin gene or the combination of information from a large number of variables (measurements) to predict the probability of AMI or CHD. The predictor information includes an assessment of genotypes and haplotypes in genomic DNA and optionally data obtainable by interviews, questionnaires, clinical examination and/or blood analyte measurements. This predictor information can be collected in any age. This method is also applicable to middle-aged persons.

The genetic, genotypic and phenotypic information used to predict AMI and CHD may relate to lipid, carbohydrate, amino acid and other nutrient (such as iron and folate) absorption, storage and metabolism, lipid transfer, oxidative and antioxidative metabolism, coagulation, fibrinolysis, platelet function, matrix proteins and degradation, blood pressure, arterial contractility and constriction, other vasoregulation, renal function, central nervous system, properties of myocardium, glucose homeostasis, adiposity, arterial and myocardial cell necrosis, apoptosis, proliferation, migration and adhesion, inflammation (such as C-reactive protein), sympathetic tone such as adrenergic receptors or human host resistance against inflammation such as the defensins.

Numerous genotyping methods have been described in the art for analysing nucleic acids for the presence of specific sequence variations e.g. SNP's, insertions and deletions (for review see Syvänen 2001 and Nedelcheva Kristensen et al. 2001). In these methods a sample containing nucleic acid (e.g. blood, tissue biopsy or buccal cells) is obtained from the patient and the sequence variations of interest are identified and assessed from the nucleic acids.

Allelic variants in genes can be discriminated by enzymatic methods (with the aid of restriction endonucleases, DNA polymerases, ligases etc.), by electrophoretic methods (e.g. single strand conformation polymorphism (SSCP), heteroduplex analysis, fragment analysis and DNA sequencing), by solid-phase assays (dot blots, microarrays, microparticles, microtiter plates etc.) and by physical methods (e.g. hybridisation analysis, mass spectrometry and denaturing high performance liquid chromatography (DHPLC)). In most of the genotyping assays different polymerase chain reaction (PCR) applications are used both to increase the signal to noise ratio as well as spare sample nucleic acid before allele discrimination. Detectable labels (fluorochromes, radioactive labels, biotin, modified nucleotides, haptens etc) can be used to enhance visualization of allelic variants.

This invention is based on the principle that one or a small number of genotypings are performed, and the mutations to be typed are selected on the basis of their ability to predict AMI and/or CHD. For this reason any method to genotype mutations in a genomic DNA sample can be used. If non-parallel methods such as real-time PCR are used, the typings are done in a row. The PCR reactions may be multiplexed or carried out separately in a row or in parallel aliquots.

The score that predicts the probability of MI or CHD may be calculated using a multivariate failure time model or a logistic regression equation as follows: Probability of coronary heart disease=[1+e^{(-(-a+Σ(bi*Xi))}]⁻¹, wherein e is Napier's constant, Xᵢ are variables related to the cardiovascular disease, bᵢ are coefficients of these variables in the logistic function, and a is the constant term in the logistic function. The model may additionally include any interaction (product) or terms of any variables Xᵢ, e.g. bᵢXᵢ. An algorithm is developed for combining the information to yield a simple prediction of MI as percentage of risk in 10 years. Alternative statistical models are a failure-time models such as the Cox's proportional hazards' model and neural networking models.

Thus, the detection method of the invention may further comprise a step of combining information concerning age, gender, the family history of hypertension, diabetes and hypercholesterolemia, and the medical history concerning cardiovascular diseases or diabetes of the subject with the results obtained from step ii) of the method (see claim 1) for confirming the indication obtained from the detection step. Said information may also concern hypercholesterolemia in the family, smoking status, CHD in the family, history of cardiovascular disease, obesity in the family, and waist-to-hip circumference ratio (cm/cm)

The detection method of the invention may also further comprise a step determining blood, serum or plasma cholesterol HDL cholesterol, LDL cholesterol, triglyceride, apolipoprotein B and Ai, fibrinogen, ferritin, transferrin receptor, C-reactive protein, serum or plasma insulin concentration.

The results from the further steps of the method as described above render possible a step of calculating the probability of a cardiovascular disease using a logistic regression equation as follows:

Probability of a cardiovascular disease=[1+e^{(-(-a+Σ(bi*Xi))}]⁻¹, where e is Napier's constant, Xᵢ are variables related to the cardiovascular disease, bᵢ are coefficients of these variables in the logistic function, and a is the constant term in the logistic function, and wherein a and bᵢ are preferably determined in the population in which the method is to be used, and Xi are preferably selected among the variables that have been measured in the population in which the method is to be used. Preferable values for bᵢ are between −20 and 20; and for i between 0 (none) and 100,000. Xᵢ are binary variables that can have values or are coded as 0 (zero) or 1 (one).

The method can be used in the prediction and early diagnosis of AMI in adult persons, stratification and selection of subjects in clinical trials, stratification and selection of persons for intensified preventive and curative interventions. The aim is to reduce the cost of clinical drug trials and health care.

The test can be applied to test the risk of developing an AMI in both
1) healthy persons, as a screening or predisposition test and
2) high-risk persons (who have e.g. family history of CHD or elevated serum cholesterol or hypertension or diabetes or any combination of these).

As inflammation is a cause of AMI and other forms of CHD, anti-inflammatory agents can plausibly be used in the prevention and treatment of AMI and chronic CHD. Persons who have a compromised host resistance to inflammation, due to e.g. reduced expression or production of human defensin proteins, will thus benefit from defensin enhancing medications, diets and other therapies. More generally, all people might benefit from the enhancement of the defensin system through a reduction of their AMI and CHD risk and consequent increase in longevity. Especially persons whose defensin levels are lowered or who have mutations in the genes encoding human defensins will benefit from such a treatment. Other groups or persons which will get increased benefit from defensin enhancing treatments are persons who already have CHD. Clinical trial testing the effect of defensing enhancement on defensin expression, body defensin levels, the progression of atherosclerosis and the incidence of AMI and other coronary events can be carried out with compounds enhancing body defensin levels and methods to measure said compounds. A method for treating a human or animal suffering from CHD or AMI by enhancing defensin availability, production or concentration in the human subject or animal may comprise an administration of a chemical entity such as a medication, a vaccination, a nutrient in natural or functional food or foodstuff, other behavioural intervention or gene therapy such as gene transfer.

As defensins are necessary in protecting against CHD and AMI, medications, dietary and other treatments that reduce human defensin levels or activity will cause adverse reactions in those persons. The likelihood of adverse reactions is the greatest in persons who already have lowered defensin levels or activities.

Transgenic animal models with mutant defensin genes and defensin gene knock-out animal models can be used to study the effect and role of defensins in the causation and progression of AMI, CHD and other diseases and conditions. RNA interference of defensin genes may be used to for the same purposes. As these model animals have increased susceptibility to CHD, they can also be used to study the efficacy and adverse reactions of any medication, nutrient or other compound in the treatment or prevention of AMI and CHD.

More particularly, the invention is directed to a method for detecting genetic variation or polymorphism, i.e. a mutation, in a defensin gene comprising the steps of:
i) providing a biological sample taken from a subject to be tested,
ii) detecting the presence or absence of a variant genotype of the defensin gene in the biological sample, the presence of a variant defensin genotype indicating an increased risk of cardiovascular disease in said subject

Preferably, genetic variation is further determined from the genes selected from the group consisting of:
a) alpha-_{2B}-adrenoceptor,
b) apolipoprotein B, and
c) beta-2-adrenergic receptor
   wherein the presence of a variant genotype in said genes indicates an increased risk of cardiovascular disease, such as myocardial infarction (AMI) or coronary heart disease (CHD), in said subject.

The method may further comprise a step of selecting a subject with a variant defensin gene sequence reducing the expression, production or levels of defensin protein for clinical drug trials testing the anticoronary and myocardial ischaemia preventing effects of compounds.

Preferably, said variant genotype of the defensin gene is a homo- or heterozygote form of the mutation.

The detection step of the method can be a DNA-assay. Such detection step can also be carried out using a gene or DNA chip, microarray, strip, panel or similar combination of more than one genes, mutations or RNA expressions to be assayed. Moreover, one of the preferable embodiments of the invention is the determination of the allelic pattern by polymerase chain reaction The detection step of the method can also be based on a capturing probe, which specifically binds to a variant defensin nucleic acid.

The biological sample for the method can be, e.g., a blood sample or buccal swab sample. From said sample genomic DNA is isolated.

The subject to be tested is preferably a mammal, more preferably a primate, and most preferably a human.

The method of the invention can be used used for determining whether a subject will benefit from treatment with a drug, nutrient or other therapy enhancing the defensin production, levels or activity or inhibiting defensin catabolism or elimination in the subject. Moreover, the method can be preferably used for determining whether a subject will be at increased risk of adverse effects or reactions if defensin antagonists are administered to a subject.

The method of the invention is preferably directed to the detection of the variants of the following genes: human beta-defensin-1 (e.g. 3′UTR+5A→G variant), human beta-defensin-129 (e.g. 5′UTR→27T→C variant and/or IVS1-13_{—}12insCTC), human alfa-defensin-5 (e.g. IVS1+198C→T variant and/or IVS1+243G→C variant), beta-2-adrenergic receptor (e.g. Gly16Arg variant and/or Glu27Gln variant) and alpha-2B-adrenergic receptor (e.g.insertion/deletion variant as defined in the Experimental Section), and apolipoprotein B gene (e.g. Thr98Ile variant). Thus, the listed gene variants are shown herein to predict CHD and/or AMI. However, a person skilled in the art may find by routine work new functional mutations in said genes. Such variants are deemed to be within the scope of those skilled in the art from the teachings herein.

The present invention also provides a method for targeting the treatment of CHD, such as angina pectoris or other form of CHD, and AMI in a subject with CHD by determining the pattern of alleles encoding a variant defensin, i.e. by determining if said subject's genotype of the defensin is of the variant type, comprising the steps presented in claim 1, and treating a subject of the variant genotype with a drug affecting defensin production or metabolism of the subject.

Another embodiment of the invention is a method for treating a human or animal suffering from CHD or AMI, said method comprising a therapy enhancing defensin availability, production or concentration of the human subject or animal, such as a mammal. Such method can be, e.g., for treating vascular complications of CHD and AMI, wherein said method may comprise a step of enhancing defensin availability, production or concentration in the circulation of a human subject or animal. The treatment may be, e.g., a dietary treatment, a vaccination, gene therapy or gene transfer. Said gene therapy may comprise a transfer of a non-variant defensin gene, such as beta-defensin-1, or fragment or derivative thereof.

The present invention further provides a kit for detecting genetic variation or polymorphism, i.e. a mutation, in a defensin gene for the determination of a risk of acute myocardial infarction, AMI, and coronary heart disease, CHD, in a subject, comprising means for defensin gene allele detection, and optionally software and/or instructions to interpret the results of the determination. The kit may also provide means for the detection of the variants of the genes selected from the group consisting of:
a) alpha-_{2B}-adrenoceptor,
b) apolipoprotein B, and
c) beta-2-adrenergic receptor

Preferably, the detected variants are the ones as described above and in the Experimental Section.

The kit can be based on a capturing nucleic acid probe specifically binding to the variant genotype as defined in the invention, and/or on a DNA chip, microarray, DNA strip, DNA panel or real-time PCR based tests. The kit may also comprise a questionnaire for obtaining patient information concerning age, gender, height, weight, the family history of hypertension and hypercholesterolemia, the medical history concerning cardiovascular diseases.

The publications and other materials used herein to illuminate the background of the invention, and in particular, to provide additional details with respect to its practice, are incorporated herein by reference.

The invention will be described in more detail in the Experimental Section.

Experimental Section

Determining Individual Genotypes

For the identification of the specific gene mentioned in the experimental section we have used Locus Link ID numbers (http://www.ncbi.nhn.nih.gov/LocusLink/). For the identification of the specific known SNPs mentioned in the experimental section we have used rs-numbers from the NCBI SNP database (http://www.ncbi.nlm.nih.gov/SNP/).

The method according to the invention for the determination of the allelic pattern of the DNA variation in question can be carried out with polymerase chain reaction (PCR) in combination with an allele specific primer extension method (SNaPshot, Applied Biosystems) followed by capillary electrophoresis with ABI Prism 3100 Genetic Analyzer (Applied Biosystems).

In a snapshot reaction the genomic DNA region containing the variation in question is amplified with PCR. The amplified PCR product is purified and used as a template in the snapshot reaction. For the snapshot reaction an extension primer is designed so that the 3′ end of the primer is immediately adjacent to the polymorphic site of interest. In the snapshot reaction the extension primer hybridizes to its complementary template in the presence of fluorescent labeled dideoxy-NThs ([F]ddNTPs) and DNA polymerase. The polymerase extends the primer by only one nucleotide, adding a single [F]ddNTP to its 3′ end Because each of the four [F]ddNTPs are labeled with different fluorescent dyes the genotypes can be discriminated.

If multiple SNPs are to be determined in the same reaction, the extension primers need to be designed so that they differ from each other significantly in length (4-6 nucleotides). The length of a primer can be modified by the addition of a variable, but a known number of non-homologous nucleotides (dT, dA, dC or cGATC) to the 5′ end of the extension primers. Due to the difference in the length of the extension primers the snapshot products can be detected in the capillary electrophoresis according to the size of the product. To perform SnaPshot genotyping under standard conditions, refer to the user manual (ABI Prism SnaPshot Multiplex kit, Protocol, Applied Biosystems).

Polymerase Chain Reaction (PCR)

The genomic DNA regions containing the mutations in question can be amplified with PCR either in separate reactions or all in one single reaction mix (i.e. multiplex PCR). The PCR amplification was conducted in a 30 μl volume: the reaction mixture contained 40 ng human genomic DNA (extracted from peripheral blood), 1×PCR Buffer (QIAGEN), 200 μM of each of the nucleotides (dATP, dCTP, dGTP, dTTP, Finnzymes), 0.75 μM of DEFB1 PCR primers, 0.5 μM of DEFB129 and DEFA5 PCR primers and 0.25 μM of ADRB2 PCR primers and 2.5 units of Hot Start Taq DNA polymerase (QIAGEN). First the reaction was hold 5 minutes at 96° C., then the following three steps were repeated for 35 cycles: 30 seconds at 94° C., 1 minute at 57° C., 1 minute at 72° C., after which reaction was kept at 72° C. for an additional 5 minutes and then hold 1 minute 10° C. and stored at 4° C. in a PTC-220 DNA Engine Dyad PCR machine (MJ Research).

For the APOB the PCR amplification was conducted in a 20 μl volume: the reaction mixture contained 40 ng human genomic DNA (extracted from peripheral blood), 1×PCR Buffer (QIAGEN), 200 μM of each of the nucleotides (dATP, dCTP, dGTP, dTTP, Finnzymes), 10 pmol of APOB PCR primers and 2.0 units of Hot Start Taq DNA polymerase (QIAGEN,); First the reaction was hold 7 minutes at 94° C., then the following three steps were repeated for 35 cycles: 45 seconds at 94° C., 45 seconds at 54° C., 1 minute at 72° C., after which the reaction was kept at 72° C. for an additional 5 minutes and then hold 1 minute 10° C. and stored at 4° C. in a PTC-220 DNA Engine Dyad PCR machine (MJ Research).

For the DEFB129 IVS1-12_{—}13insCTC the amplification was conducted in a 40 μl volume: the reaction mixture contained 60 ng human genomic DNA (extracted from peripheral blood), 1×PCR Buffer (QIAGEN), 200 μM of each of the nucleotides (dATP, dCTP, dGTP, dTTP, Finnzymes), 20 pmol of DEFB 129 IVS 1-12_{—}13insCTC PCR primers and 3.0 units of Hot Start Taq DNA polymerase (QIAGEN,). First the reaction was hold 7 minutes at 96° C., then the following three steps were repeated for 35 cycles: 45 seconds at 94° C., 45 seconds at 57° C., 1 minute at 72° C., after which the reaction was kept at 72° C. for an additional 5 minutes and then hold 1 minute 10° C. and stored at 4° C. in a PTC-220 DNA Engine Dyad PCR machine (MJ Research).

The nucleotide sequence of the PCR primer pair for the amplification of the human DEFB1 gene (defensin beta 1, Locus link ID: 1672) 3′UTR+5A→G mutation (rs1047031) was as follows: 5′-CAT AAT TTC AGC CCG ATG TG-3′ (SEQ ID NO: 1) and 5′-CAC CCT AAC CCC CTA CTT CT-3′ (SEQ ID NO:2).

The nucleotide sequence of the PCR primer pair for the amplification of the human DEFB129 gene (defensin beta 129, Locus link ID: 140881) 5′UTR−27T>C (rs2298148) was as follows: 5′-GGG CTT GCT CTT TCT TTC-3′ (SEQ ID NO:3) and 5′-TCC TTG GTT CCT CTC ATC-3′ (SEQ ID NO:4).

The nucleotide sequence of the PCR primer pair for the amplification of the human ADRB2 gene (Beta-2-adrenergic receptor, Locus link ID: 154) Gly16Arg (rs1042713) and Glu27Gln (rs1042714) mutations was as follows: 5′- CTG AGT GTG CAG GAC GAG-3′ and (SEQ ID NO:5) 5′-CAC ATT GCC AAA CAC GAT-3′ (SEQ ID NO:6).

The nucleotide sequence of the PCR primer pair for the amplification of the human DEFA5 gene (defensin alpha 5, Locus link ID: 1670) IVS1+198C>T (in the following sequence, [SEQ ID NO:7, SEQ ID NO:8], the DEFA5 IVS+198C>T substitution is located at the position 553) and the IVS1+243G>C variants (in the following sequence, [SEQ ID NO:7, SEQ ID NO:8], the DEFA5 IVS+243G>C substitution is located at the position 598) was as follows: 5′-AGA AAG AGG AGC ATC AAA G-3′ (SEQ ID NO:9) and 5′-TCA AGC CTA TTA GCC TAC A-3′ (SEQ ID NO:10).

The nucleotide sequence of the PCR primer pair for the amplification of the human APOB gene (apolipoprotein B, Locus link ID: 338) Thr98Ile mutation (also known as Thr71Ile mutation, rs1367117) was as follow: 5′-GAC AAC CTC AAT GCT CTG CT-3′ (SEQ ID NO:11) and 5′-TGA CTT ACC TGG ACA TGG CT-3′ (SEQ ID NO:12).

The nucleotide sequence of the PCR primer pair for the amplification of the human DEFB 129 gene (defensin beta 129, Locus link ID:140881) IVS1-12_{—}13insertionCTC variant (in the following sequence, SEQ ID NO:32, SEQ ID NO:33 the insertion is in position 444-46) was as follow: 5′-GGC TAC TGA GTT TGG TGA-3′ (SEQ ID NO:34) and 5′-GTG TIT ATT GAA TGA CTG ATG-3′ (SEQ ID NO:35).

The PCR products were purified with SAP (Shrimp Alkaline Phosphatase, USB) and ExoI (Exonuclease I, New England Biolabs) treatment This was done to avoid the participation of the unincorporated dNTPs and primers from the PCR reaction to the subsequent primer-extension reaction. More specifically, 2.5 μl of SAP (1 unit/μl, USB), 0.25 μl of ExoI (20 units/μl, New England Biolabs), 1.0 μl of 10× ExoI buffer (New England Biolabs) and 6.25 μl H₂O were added to 5 μl of the PCR product Reaction was mixed and incubated at 37° C. for 1 hour. After that the reaction was kept at 75° C. for 15 minutes to inactivate the enzymes and stored at 4° C.

In the subsequent primer extension reaction (SNaPshot reaction) 1.5 μl of SNaPshot Multiplex Ready Reaction Mix (Applied Biosystems), 3 μl of purified PCR products, 1 μl of pooled extension primers (1 μM each) and 4.5 μl buffer (1× AmpliTaq Gold buffer 2 mM MgCl₂, Applied Biosystems) are mixed in a tube. The reaction is incubated at 96° C. for 5 seconds and then subject to 35 cycles of 95° C. for 10 s, 50° C. for 5 s and 60° C. for 30 s in a PTC-220 DNA Engine Dyad PCR machine (MJ Research).

The nucleotide sequence of the extension primer for the genotyping of human DEFA5 IVS1+198C>T mutation in a SNaPShot reaction was: 5′-TTT TTT TTT TTT TTT CTT TTT TCT AAG ACT TTC AG-3′ (SEQ ID NO:13).

The nucleotide sequence of the extension primer for the genotyping of human DEFA5 IVS1+243G>C mutation in a SNaPShot reaction was: 5′-TTT TTT TTT TTT TTT TTT TGC TAC TTT TAA GAT AGA AAG A-3′ (SEQ ID NO:14).

The nucleotide sequence of the extension primer for the genotyping of the human DEFB1 3′UTR+5A>G mutation in a SNaPShot reaction was: 5′-TTT TTT TTT TTT TTT TTT TTT TTT TTT AGT GCT GCA AGT GAG CTG-3′ (SEQ ID NO: 15).

The nucleotide sequence of the extension primer for the genotyping of human DEFB129 IVS1-27T>C mutation in a SNaPshot reaction was: 5′-TTT TTT TTT TTT TTT TTT TTT TTT TTT TTT TTT CCA GAG AGG AAG CCT TG-3′ (SEQ ID NO:16).

The nucleotide sequence of the extension primer for the genotyping of human ADRB2 Gly16Arg mutation in a SNaPShot reaction was: 5′-T TTT TTT TTT TTT TTT TTT TTT TTT TTT TTT TTT TTT TTC TTG CTG GCA CCC AAT-3′ (SEQ ID NO:17).

The nucleotide sequence of the extension primer for the genotyping of human ADRB2 Glu27Gln mutation in a SNaPShot reaction was: 5′-TTT TTT TTT TTT TTT TTT TTT TTT TTT TTT TTT TTT TTT TTT TAC CAC GAC GTC ACG CAG-3′ (SEQ ID NO:18).

The nucleotide sequence of the extension primer for the genotyping of human APOB Thr98Ile (Thr71Ile, rs1367117) mutation in a SNaPShot reaction was: 5′-TTT TTT TTT TTT TGA AGA CCA GCC AGT GCA-3′ (SEQ ID NO:19).

The nucleotide sequence of the extension primer for the genotyping of human DEFB129 gene (defensin beta 129) IVS1-12_{—}13insertionCTC variant in a SNaPshot reaction was: 5′-TTT TTT TTT TTT TTT TTT TTT TTT TTT TTT TTT GCT CAA TGG CTT TCT CT-3′ (SEQ ID NO:56). In the snapshot reaction the deletion CTC allele is detected as nucleotide T whereas the presence of the insertion CTC allele is detected as nucleotide C.

After the primer extension reaction (snapshot reaction) 1 unit of SAP (USB) was added to the reaction mix and the reaction was incubated at 37° C. for 1 hour. The enzyme was inactivated by incubating the reaction mix at 75° C. for 15 minutes and placed at 4° C. The post-extension treatment was done to prevent the unincorporated fluorescent ddNTPs obscuring the primer extension products (SNaPshot products) during electrophoresis with ABI Prism 3100 Genetic Analyzer.

DNA Fragment Analysis of ADRA2B Insertion/Deletion Polymorphism

The insertion/deletion polymorphism of ADRA2B gene concerns an insertion or a deletion of three glutamic acids (Glu) in the region of 12 Glu amino acids in the codons 298-309. Thus depending on the allele, there is either 9 Glu (deletion, variant form) (SEQ ID NO:20) or 12 Glu (insertion) (SEQ ID NO:22) at the ADRA2B locus. Depending on whether the amplified allele had an insertion or a deletion in the studied locus, the size of the PCR product was 91 bp (insertion allele) or 82 bp (deletion allele). For homotzygotes (insertion/insertion or deletion/deletion) only one size of a fragment was detected either 91 bp or 82 bp, respectively. For heterotzygotes both of the above mentioned fragments were detected.

The PCR amplification was conducted in a 20 μl volume: the reaction mixture contained 40 ng human genomic DNA (extracted from peripheral blood), 1×PCR Buffer (QIAGEN), 200 μM of each of the nucleotides (dATP, dCTP, dGTP, dTTP), 10 pmol of ADRA2B PCR primers and 2.0 units of Hot Start Taq DNA polymerase (QIAGEN). First the reaction was hold 7 minutes at 95° C., then the following three steps were repeated for 35 cycles: 45 seconds at 94° C., 45 seconds at 54° C., 1 minute at 72° C., after which the reaction was kept at 72° C. for an additional 5 minutes and then hold 1 minute 10° C. and stored at 4° C. in a PTC-220 DNA Engine Dyad PCR machine (MJ Research).

The PCR primer pair for the amplification of the ADRA2B gene (alpha-2B-adrenergic receptor, Locus link ID: 151) insertion/deletion polymorphism was as follows 5′-GGG TGT TTG TGG GGC ATC TC-3′ (SEQ ID NO:24) and 5′-TGG CAC TGC CTG GGG TTC A-3′ (SEQ ID NO:25). A fluorescent label has been added to the 5′ end of one of the above mentioned PCR primers. Therefore, the PCR fragment is detectable in the capillary electrophoresis conducted with ABI Prism 3100 Genetic Analyzer (Applied Biosystems).

Capillary Electrophoresis with ABI Prism 3100 Genetic Analyzer

Aliquots of 1 μl of pooled SNaPshot products, 0.5 μl ADRA2B PCR product, 9.25 μl of Hi-Di formamide (Applied Biosystems) and 0.25 μl GeneScan-120 LIZ size standard (Applied Biosystems) were combined in a 96-well 3100 optical microamp plate (Applied Biosystems). The reactions were denatured by placing them at 95° C. for 5 minutes and then loaded onto a ABI Prism 3100 Genetic Analyzer (Applied Biosystems). Electrophoresis data was processed and the genotypes were visualized by using the GenoTyper Analysis Program version 3.7 (Applied Biosystems).

Identification of New Mutations in Human Beta-Defensin Genes

We used the hierarchial phenotype-targeted sequencing method (see WO 02/074230) to find new mutations in the beta-defensin-1 gene. As defensins are known to act to protect against infections, it was hypothesised that subjects with frequent infections would have lowered and subjects with infrequent infections would have high or normal body defensin levels and activities. Forty-eight Kuopio Ischaemic Heart Disease Risk Factor Study (KIHD) examinees with the largest number of respiratory and urinary infections in the previous five years and 48 gender- and age-matched subjects with neither respiratory nor urinary infections in the previous five years were selected for sequencing. We sequenced five different Defensin Alpha genes (DEFA1, DEFA3, DEFA4, DEFA5 and DEFA6) and six different Defensin Beta genes (DEFB1, DEFB103, DEFB4, DEFB118, DEFB126 and DEFB129).

In sequencing we found five mutations in DEFA5 gene (DEFA5 IVS1+198C>T, DEFA5 IVS1+243G>C, DEFA5 Arg71Cys [rs7839771], DEFA5 3′UTR+109A>G and DEFA5 3′-UTR+168C>T).

In DEFB1 gene we found five mutations (DEFB1 5′UTR−52G>A [rs1799946], DEFB1 5′UTR−44C>G [rs1800972], DEFB1 5-UTR−20A>G [rs11362], DEFB1 IVS1+19T>A [rs2293958] and DEFB1 3′UTR+5A>G [rs1047031]).

In DEFB2 gene we found three mutations (DEFB2 5′UTR−108t>C [rs2740086], DEFB2 T>C Pro29Pro [rs2740090] and DEFB2 3′UTR+164G>A [rs2737531]).

From DEFB118 gene we found one mutation (DEFB118 T>C Cys34Arg).

In DEFB126 gene we found two mutations (DEFB126 deletion CAAA163_{—}166 frameshift and DEFB126 deletion CC317_{—}318 frameshift).

In DEFB129 gene we found five mutations (DEFB129 5′UTR−41G>A [rs2298149], DEFB129 5′UTR−27T>C [rs2298148], DEFB129 IVS1-68C>T [rs6074833], DEFB129 IVS1-13_{—}12insertionCTC and DEFB129 A201G synonymous to Leu67Leu).

Of the above mentioned Defensin Alpha and Defensin Beta gene variants the following 9 (nine) have not been reported previously: DEFA5 IVS1+198 C>T, DEFA5 IVS1+243 G>C, DEFA5 3′UTR+109 A>G, DEFA5 3′UTR+168 C>T, DEFB129 IVS1-12 insertion deletion CTC, DEFB129 A>G leu67leu (CTG67CTA), DEFB118 T>C Cys34Arg (TG34CGC), DEFB126 exon 2 deletion c.163_{—}166delCAAA and DEFB126 exon 2 deletion c.317_{—}318delCC.

The nucleotide sequence of the PCR primer pair for the amplification of the human DEFA5 gene (defensin alpha 5) IVS1+198 C>T variant (in the following sequence, [SEQ ID NO:7, SEQ ID NO:8)] the substitution is located at the position 553) was as follow: 5′-AGA AAG AGG AGC ATC AAA G-3′ (SEQ ID NO:9) and 5′-TCA AGC CTA TTA GCC TAC A-3′ (SEQ ID NO:10). The sequencing primer was: 5′-TCA GGT CTT CTC CCA GCA (SEQ ID NO:26)

The nucleotide sequence of the PCR primer pair for the amplification of the human DEFA5 gene (defensin alpha 5, Locus link ID:1670) IVS1+243 G>C variant (in the following sequence, [SEQ ID NO:7, SEQ ID NO:8)] the substitution is located at the position 598) was as follow: 5′-AGA AAG AGG AGC ATC AAA G-3′ (SEQ ID NO:9) and 5′-TCA AGC CTA TTA GCC TAC A-3′ (SEQ ID NO:10). The sequencing primer was: 5′-TCA GGT CTT CTC CCA GCA (SEQ ID NO:26)

The nucleotide sequence of the PCR primer pair for the amplification of the human DEFA5 gene (defensin alpha 5, Locus link ID:1670) 3′UTR+109 A>G variant (in the following sequence, [SEQ ID NO:27, SEQ ID NO:28] the substitution is located in position 515) was as follow: 5′-GGA TGA AGC AGA ATG AAG A-3′ (SEQ ID NO:29) and 5′-AAA GGA ACC ATA CAA ACC A-3′ (SEQ ID NO:30). The sequencing primer was: 5′-GTT AGT CTG GCT GTG CTT-3′ (SEQ ID NO:31).

The nucleotide sequence of the PCR primer pair for the amplification of the human DEFA5 gene (defensin alpha 5, Locus link ID:1670) 3′UTR+168 C>T variant (in the following sequence, [SEQ ID NO:27, SEQ ID NO:28] the substitution is located in position 574) was as follow: 5′-GGA TGA AGC AGA ATG AAG A-3′ (SEQ ID NO:29) and 5′-AAA GGA ACC ATA CAA ACC A-3′ (SEQ ID NO:30). The sequencing primer was: 5′-GTT AGT CTG GCT GTG CTT-3′ (SEQ ID NO:31).

The nucleotide sequence of the PCR primer pair for the amplification of the human DEFB129 gene (defensin beta 129, Locus link ID:140881) IVS1-12_{—}13insertionCTC variant (in the following sequence, SEQ ID NO:32, the insertion is in position 444-446) (SEQ ID NO:33) was as follow: 5′-GGC TAC TGA GTT TGG TGA-3′ (SEQ ID NO:34) and 5′-GTG TTT ATT GAA TGA CTG ATG-3′ (SEQ ID NO:35). The sequencing primer was: 5′-CAA GGA AGG CAG ACT AAA-3′ (SEQ ID NO:36).

The nucleotide sequence of the PCR primer pair for the amplification of the human DEFB129 gene (defensin beta 129, Locus link ID:140881) leu67leu (CTA67CTG), A>G variant (SEQ ID NO:37) (SEQ ID NO:39) was as follow: 5′-GGC TAC TGA GTT TGG TGA-3′ (SEQ ID NO:34) and 5′-GTG TTT ATT GAA TGA CTG ATG-3′ (SEQ ID NO:35). The sequencing primer was: 5′-CAA GGA AGG CAG ACT AAA-3′ (SEQ ID NO:36).

The nucleotide sequence of the PCR primer pair for the amplification of the human DEFB118 gene (defensin beta 118, Locus link ID:117285) Cys34Arg (TGC34CGC), T>C mutation (SEQ ID NO:41) (SEQ ID NO:43) was as follow: 5′-AGG TTG AGT ATT TGC CAG AC-3′ (SEQ ID NO:45) and 5′-AGG ACA GGG GTG AGT GAT A-3′ (SEQ ID NO:46). The sequencing primer was: 5′-AGG TTG AGT ATT TGC CAG AC -3′ (SEQ ID NO:45).

The nucleotide sequence of the PCR primer pair for the amplification of the human DEFB126 (defensin beta 126, Locus link ID:81623) exon 2 deletion c.163_{—}166delCAAA (SEQ ID NO:47) (SEQ ID NO:49) was as follow: 5′-AAT GGT GAG AAA GAT GAC AG-3′ (SEQ ID NO:51) and 5′-GTT GAA TGG AGG GAA AGT-3′ (SEQ ID NO:52). The sequencing primer was: 5′-GTA GGT ATT TAT GAT TAG-3′ (SEQ ID NO:53). This mutation leads to a change in protein amino acid structure of the DEFB126 gene from the amino acid codon 55 and finally to a premature STOP codon in amino acid position 82 (SEQ ID NO:47).

The nucleotide sequence of the PCR primer pair for the amplification of the human DEFB126 gene (defensin beta 126, Locus link ID:81623) exon 2 deletion c.317_{—}318delCC (SEQ ID NO:54) (SEQ ID NO:49) was as follow: 5′-AAT GGT GAG AAA GAT GAC AG-3′ (SEQ ID NO:51) and 5′-GTT GAA TGG AGG GAA ACT-3′ (SEQ ID NO:52). The sequencing primer was: 5′-GTA GGT ATT TAT GAT TAG-3′ (SEQ ID NO:53). This mutation also leads to an altered amino acid structure of the DEFB126 gene from the amino acid codon 106 (SEQ ID NO:54).

Testing the Risk of AM and CHD

Risk factors for MI and coronary heart disease were studied in the KIHD cohort. Briefly, the “Kuopio Ischaemic Heart Disease Risk Factor Study” (KIHD) is a prospective population study in men in Eastern Finland (Salonen 1988, Tuomainen et al. 1999). The study protocol for KIHD was approved by the Research Ethics Committee of the University of Kuopio. The study sample comprised men from Eastern Finland aged 42, 48, 54 or 60 years. A total of 2682 men were examined during 1984-89. All participants gave a written informed consent The follow-up of coronary events was to the end of 2001, providing an average follow-up time of 14.4 years. Genotypings were carried out for approximately 1600 men, resulting to over 23,000 person-years of follow-up.

Data on CHD and AM during the follow-up were obtained by computer record linkage to the national computerized hospital discharge registry. Diagnostic information was collected from the hospitals and all heart attacks events were classified according to rigid predefined criteria The diagnostic classification of acute coronary events was based on symptoms, electrocardiographic findings, cardiac enzyme elevations, autopsy findings and the history of CHD. Each suspected coronary event (ICD-9 codes 410-414 and ICD-10 codes I20-I25) was classified into 1) a definite acute myocardial infarction (AMI), 2) a probable AMI, 3) a typical acute chest pain episode of more than 20 minutes indicating CHD, 4) an ischemic cardiac arrest with successful resuscitation, 5) no acute coronary event or 6) an unclassifiable fatal case. The categories 1) to 3) were combined for the present analysis to denote MI. Of 1548 male subjects with complete data, used in the analysis, 256 men developed an AMI during the follow-up.

Hypertension was defined as either systolic blood pressure (BP)≧165 mmHg or diastolic BP≧95 mmHg or antihypertensive treatment Both blood pressures were measured in the morning by a nurse with a random-zero mercury sphygmomanometer. The measuring protocol included three measurements in supine, one in standing and two in sitting position with 5-minutes intervals. The mean of all six measurements were used as systolic and diastolic blood pressures. Family history of CHD was defined positive if either the subject's mother, father or a sibling had a history of AMI or angina pectoris. Family histories of cerebrovascular stroke and diabetes were defined similarly. Adulthood socioeconomical status (SES) is an index comprised of measures of education, occupation, income and material living conditions. The scale is inverse, low score corresponding to high SES. These data have been collected by a self administered questionnaire. Serum ferritin was assessed with a commercial double antibody radioimmunoassay (Amersham International, Amersham, UK). Lipoproteins, including high density lipoprotein (HDL) and low density lipoprotein (LDL), were separated from fresh serum samples by ultracentrifugation followed by direct very low density lipoprotein (VLDL) removal and LDL precipitation Cholesterol concentration was then determined enzmically Serum C-reactive protein was measured by a commercial high-sensitive immunometric assay (Immulite High Sensitivity CR Assay, DPC, Los Angeles). Genotyping of the paraoxonase 1 and HFE (HLA-H) mutations have been described elsewhere (Salonen et al. 1999, Tuomainen et al 1999).

In the beta-defensin 1 gene, 3′UTR+5, of the 1548 men genotyped, 165 were AA homozygotes, 690 heterozygotes and 693 GG homozygotes. Of the GG homozygotes, 19.0% (132 men) developed their first AMI during the follow-up, as compared with 14.5% (124 men) of the other men (odds ratio 1.39, 95% CI 1.06 to 1.82, p=0.017). In a multivariate logistic model controlling for the strongest other covariates, the respective adjusted odds ratio was 1.35 (95% CI 1.01 to 1.80, p=0.044, Table 1). The association between the GG genotype and the risk of AMI tended to be stronger among men who had no prior history of CHD (odds ratio 1.44, 95% CI 1.04 to 2.00, p=0.030) than among those who had prior CHD (odds ratio 1.32, 95% CI 0.81 to 2.17, p=0.314).

Other gene mutations that predicted AMI in the logistic model were the deletion/insertion in the alpha-2B-adrenergic receptor gene and the Thr98Ile SNP in the apolipoprotein B gene (Table 1). Phenotypic data that added to the prediction of AMI were age, history of any atherosclerotic disease, cigarette-years of smoking, family history of CHD and diabetes, the presence of type 2 diabetes, and serum total and high-density lipoprotein (HDL) cholesterol (Table 1). Of these 12 variables, an empirical binary logistic function was constructed (Table 1). The population attributable risk, calculated across quintiles of the risk score, according to Miettinen O S, was 0.76. Odds ratios for quintiles (the lowest as reference): 12.8, 95% confidence interval (CI) 7.2 to 22.9, 6.4 (3.5 to 11.5), 2.4 (1.3 to 4.6) and 1.5 (0.8 to 3.1). When a split at the predicted probability (score value) of 0.2 was used, the odds ratio was 5.3 with 95% CI 4.0 to 7.0, p<0.001.

We also analyzed the prediction by gene mutations and phenotypic data the risk of AMI within five years of the baseline examination (Table 2). Another beta-defensin (DEFB129) SNP, located in IVS1-13_{—}12insCTC, was a strong predictor of AMI. The carriers of the insertion CTC allele had 2.3-fold risk of AMI (95% Cl 1.4 to 3.9, p=0.002). Also the apolipoprotein B Thr homozygosity predicted AMI strongly, and the deletion homogenicity of alpha-2B-adrenergic receptor gene fairly strongly. Phenotypic data that predicted AMI in five years were age, history of any atherosclerotic disease, cigarette-years of smoking, the presence of hypertension, the use of cholesterol lowering medication, family history of CHD and diabetes, waist-to-hip circumference ratio, and serum concentrations of total and high-density lipoprotein HDL) cholesterol and ferritin. When the default split of the predicted probability (0.50) was used, the model predicted correctly 95.5% of the observed AMIs. When a split at the predicted probability of 0.2 was used, the odds ratio was 11.1 with 95% CI 5.9 to 21.2, p<0.001.

We also analyzed the predictors of AMI in men who had a family history of CHD (Table 3). The same three mutations predicted AMI Of the measurements by questionaire the strongest predictors were the history of CHD in the subject and his socioeconomic status. Of the biochemical measurements, the most predictive were serum ferritin copncentration (classified into two categories), serum C-reactive protein, serum LDL cholesterol and serum HDL cholesterol (protective). When the default split of the predicted probability (0.50) was used, the model predicted correctly 94.0% of the observed AMIs. When a split at the predicted probability of 0.2 was used, the odds ratio was 8.2 with 95% CI 4.0 to 16.8, p<0.001.

In another statistical analysis, we analyzed the predictors of AMI within two years of risk factor measurements (Table 4). The Leu54Met mutation in the paraoxonase 1 gene and Cys282Tyr mutation in the BPE (HLA-H) gene were the strongest genetic predictors of AMI. Other, non-genetic predictors are presented in Table 4.

Thus, we disclose here a novel genetic test based on genotyping mutations in a human defensin gene, such as human beta-defensin 1 and 129 genes, with an optional multivariable model that predicts future myocardial infarction very well in the data set they were derived of. On the basis of our invention and empirical evidence supporting it, mutations in the human beta-defensins are associated with an increased risk of AMI and CHD both in healthy persons and in those who have a family history of CHD. Thus, for the first time it is showed that defensins are related to AMI and CHD and a mutation in a defensin gene can be a statistically significant risk factor for AMI and CHD.

When information of a few important mutations is combined with phenotypic information, the prediction of a multivariate risk prediction model is enhanced An advantage is that only a small number of genotypings and biochemical or other measurements need to be carried out and a very short self-administered questionnaire needs to be filled in. The risk model can be estimated/constructed for different lenghts of follow-up, enabling the use of them for different purposes.

TABLE 1
A multivariate logistic model predicting the risk of MI in 1548
men in 9-15 years (256 experienced an AMI during the follow-up).

| | | | | | | 95% |
|---|---|---|---|---|---|---|
| | | | | | | Confidence |
| Predictor | Mutation | Coefficient (b₁) | S.E. | p-value | Odds ratio | interval |
| Beta-Defensin 1 (GG hozygote vs. other) | 3′UTR + 5 A/G | 0.30 | 0.15 | 0.044 | 1.35 | 1.01, 1.80 |
| Alpha-2B-adrenergic receptor (deletion carrier | Insertion/deletion | 0.56 | 0.21 | 0.007 | 1.75 | 1.16, 2.65 |
| vs. non-carrier) | | | | | | |
| Alpha-2B-adrenergic receptor (I/D | Insertion/deletion | 0.31 | 0.18 | 0.088 | 1.36 | 0.96, 1.94 |
| heterozygote vs. non-carrier) | | | | | | |
| Apolipoprotein B (Thr homozygote vs. other) | Thr98Ile (Thr71Ile) | 0.49 | 0.27 | 0.067 | 1.63 | 0.97, 2.74 |
| Age (per year) | NA | 0.08 | 0.016 | <0.001 | 1.08 | 1.05, 1.12 |
| History of atherosclerotic disease (yes vs. no) | NA | 0.69 | 0.16 | <0.001 | 1.99 | 1.45, 2.72 |
| Cigarette-years (per cigarettes/d multiplied by | NA | 0.001 | <0.001 | 0.001 | 1.001 | 1.00, 1.001 |
| years smoked) | | | | | | |
| CHD in the family (yes vs. no) | NA | 0.64 | 0.15 | <0.001 | 1.90 | 1.41, 2.56 |
| Diabetes in the family (yes vs. no) | NA | 0.48 | 0.16 | 0.002 | 1.62 | 1.19, 2.21 |
| Diabetes in the subject (yes vs. no) | NA | 1.23 | 0.29 | <0.001 | 3.41 | 1.93, 6.04 |
| Serum total cholesterol (per 1.0 mmol/L) | NA | 0.22 | 0.07 | 0.001 | 1.25 | 1.09, 1.44 |
| Serum HDL cholesterol (per 1.0 mmol/L) | NA | −0.84 | 0.27 | 0.002 | 0.43 | 0.26, 0.73 |

Constant 9.784.

TABLE 2
A multivariate logistic model predicting the risk of MI in 1548 men
in 5 years (of whom 69 experienced an AMI during the follow-up).

| | | | | | | 95% |
|---|---|---|---|---|---|---|
| | | | | | | Confidence |
| Predictor | Mutation | Coefficient (b₁) | S.E. | p-value | Odds ratio | interval |
| Beta-Defensin 129 (insertion CTC carrier vs. | IVS1- | 0.831 | 0.268 | 0.002 | 2.30 | 1.36, 3.88 |
| other) | 13_12insCTC | | | | | |
| Alpha-2B-adrenergic receptor (deletion | Insertion/deletion | 0.295 | 0.298 | 0.321 | 1.34 | 0.75, 2.41 |
| homozygote vs. other) | | | | | | |
| Apolipoprotein B (Thr homozygote vs. other) | Thr98Ile (Thr71Ile) | 1.227 | 0.374 | 0.001 | 3.41 | 1.64, 7.10 |
| Age (per year) | NA | 0.078 | 0.032 | 0.016 | 1.081 | 1.02, 1.15 |
| History of atherosclerotic disease (yes vs. no) | NA | 0.766 | 0.277 | 0.006 | 2.15 | 1.25, 3.70 |
| Cigarette-years (per cigarettes/d multiplied by | NA | 0.072 | 0.035 | 0.037 | 1.08 | 1.004, 1.15 |
| years smoked) divided by 100 | | | | | | |
| Hypertension (yes vs. no) | NA | 0.447 | 0.277 | 0.107 | 1.56 | 0.91, 2.69 |
| Waist-to-hip circumference ratio (m/cm) | NA | 0.024 | 0.018 | 0.186 | 1.02 | 0.99, 1.06 |
| CHD in the family (yes vs. no) | NA | 0.843 | 0.285 | 0.003 | 2.32 | 1.33, 4.06 |
| Diabetes in the family (yes vs. no) | NA | 0.352 | 0.276 | 0.202 | 1.42 | 0.83, 2.44 |
| Cholesterol lowering medication (yes vs. no) | NA | 1.713 | 0.844 | 0.042 | 5.55 | 1.06, 29.0 |
| Serum total cholesterol (per 1.0 mmol/L) | NA | 0.174 | 0.118 | 0.143 | 1.19 | 0.94, 1.50 |
| Serum HDL cholesterol (per 1.0 mmol/L) | NA | −0.818 | 0.512 | 0.110 | 0.44 | 0.16, 1.20 |
| Serum ferritin (per 100 micrograms/L) | NA | 0.131 | 0.062 | 0.034 | 1.14 | 1.01, 1.29 |

Constant term 14.144. For adjustment purpose, the model also included a term for examination month. The models predicted correctly 95.5% of the observed acute myocardial infarctions (p < 0.001).

TABLE 3
A multivariate logistic model predicting the 5-year risk of MI in 761 men with
a family history of CHD (of whom 49 experienced an AMI during the follow-up).

| | | | | | | 95% |
|---|---|---|---|---|---|---|
| | | | | | | Confidence |
| Predictor | Mutation | Coefficient (b₁) | S.E. | p-value | Odds ratio | interval |
| Beta-Defensin 129 (insertion CTC carrier vs. | IVS1- | 0.555 | 0.327 | 0.090 | 1.74 | 0.92, 3.30 |
| other) | 13_12insCTC | | | | | |
| Alpha-2B-adrenergic receptor (deletion | Insertion/deletion | 0.916 | 0.339 | 0.007 | 2.50 | 1.29, 4.86 |
| homozygote vs. other) | | | | | | |
| Apolipoprotein B (Thr homozygote vs. other) | Thr98Ile (Thr71Ile) | 1.151 | 0.469 | 0.014 | 3.16 | 1.26, 7.93 |
| History of CHD (yes vs. no) | NA | 1.220 | 0.337 | <0.001 | 3.39 | 1.75, 6.56 |
| Socioeconomic status (score of 0 to 23) | NA | 0.079 | 0.042 | 0.063 | 1.08 | 1.00, 1.18 |
| Hypertension (yes vs. no) | NA | 0.492 | 0.328 | 0.134 | 1.64 | 0.86, 3.11 |
| Serum LDL cholesterol (per 1.0 mmol/L) | NA | 0.190 | 0.156 | 0.224 | 1.21 | 0.89, 1.64 |
| Serum HDL cholesterol (per 1.0 mmol/L) | NA | −0.896 | 0.644 | 0.164 | 0.41 | 0.12, 1.44 |
| Serum C-reactive protein (mg/L) | | 0.063 | 0.038 | 0.097 | 1.07 | 0.99, 1.15 |
| Serum ferritin (>200 micrograms/L vs. less) | NA | 1.017 | 0.336 | 0.002 | 2.77 | 1.43, 5.34 |

Constant term 17.612. For adjustment purpose, the model also included a term for examination month. The models predicted correctly 94.0% of the observed acute myocardial infarctions (p < 0.001).

TABLE 4
A multivariate logistic model predicting the 2-year risk of MI in
1587 men (of whom 31 experienced an AMI during the follow-up).

| | | | | | | 95% |
|---|---|---|---|---|---|---|
| | | | | | | Confidence |
| Predictor | Mutation | Coefficient (b₁) | S.E. | p-value | Odds ratio | interval |
| Paraoxonase 1 | Leu54Met | 1.400 | 0.438 | 0.001 | 4.06 | 1.72, 9.57 |
| HFE | Cys282Tyr | 0.499 | 0.590 | 0.398 | 1.65 | 0.52, 5.24 |
| History of prior AMI (yes vs. no) | NA | 1.298 | 0.581 | 0.025 | 3.66 | 1.17, 11.43 |
| History of claudication (yes vs. no) | NA | 1.311 | 0.574 | 0.022 | 3.71 | 1.20, 11.44 |
| Antihypertensive medication (yes vs. no) | NA | 0.755 | 0.453 | 0.095 | 2.13 | 0.88, 5.16 |
| Family history of cerebrovascular stroke (yes vs. no) | NA | 0.894 | 0.411 | 0.030 | 2.45 | 1.09, 5.47 |
| Waist-to-hip circumference ratio (m/cm) | NA | 0.040 | 0.023 | 0.081 | 1.04 | 1.00, 1.09 |
| Serum cholesterol (per 1.0 mmol/L) | NA | 0.310 | 0.162 | 0.056 | 1.36 | 0.99, 1.87 |
| Serum ferritin (>200 micrograms/L vs. less) | NA | 0.932 | 0.393 | 0.018 | 2.54 | 1.18, 5.48 |

Constant term 30.575. For adjustment purpose, the model also included a term for examination month. The models predicted correctly 98.1% of the observed acute myocardial infarctions (p < 0.001).

### REFERENCES

Bamathan E S, Raghunath P N, Tomaszewski J E, Ganz T, Cines D B, Higazi A al-R Immuohistochemical localization of defensin in human coronary vessels. Am J Pathol. 1997; 150: 1009-20.

Bensch K W, Raida M, Magert H J, Schulz-Knappe P, Forssmann W G. hBD-1: a novel beta-defensin from human plasma FEBS Lett. 1995; 368: 331-5.Callen D F, Baker E, Simmers R N, Seshadri R, Roninson D B. Localization of the human multiple drug resistance gene, MDR1, to 7q21.1. Hum. Genet. 1987; 77: 142-144.

Diverse Populations Collaborative Group. Prediction of mortality from coronary heart disease among diverse populations: is there a common predictive function? Heart 2002; 88: 222-8.

Dork T, Stuhrmann M. Polymorphisms of the human beta-defensin-1 gene. Mol Cell Probes. 1998; 12: 171-3.

Ganz T, Lehrer R I. Defensins. Pharmacol Ther. 1995; 66: 191-205.

Hoover D M, Chertov O, Lubkowski J. The structure of human beta-defensin-1: new insights into structural properties of beta-defensins. J Biol Chem. 2001; 276: 39021-6.

Jia B P, Schutte B C, Schudy A, Linzmeier R, Guthmiller J M, Johnson G K, et al. Discovery of new human beta-defensins using a genomics-based approach. Gene. 2001; 263: 211-8.

Lehmann J, Retz M, Harder J, Krams M, Kellner U, Hartmann J, et al. Expression of human beta-defensins 1 and 2 in kidneys with chronic bacterial infection. BMC Infect Dis. 2002; 2: 20.

Nedelcheva Kristensen V, Kelefiotis D, Kristensen T and Borresen-Dale: High-Throughput methods for detection of genetic variation Biotechniques 30:318-332, 2001.

Salonen J T. Is there a continuing need for longitudinal epidemiologic research—The Kuopio Ischaemic Heart Disease Risk Factor Study. Ann Clin Res 1988: 20: 46-50.

Salonen J T, Malin R. Tuomainen T-P, Nyyssönen K, Nissinen T, Lakka T A, Lehtimäki T. Polymorphism in the high density lipoprotein paraoxonase gene and the risk of acute myocardial infarction in men: a prospective population-based study. Brit Med J 1999; 319: 487-9.

Schutte B C, McCray P B Jr. β-defensins in lung host defense. Annu Rev Physiol. 2002; 64: 709-48.

Snapir A, Heinonen P, Tuomainen T-P, Alhopuro P, Karvonen M K, Lakka T A, Nyyssönen K, Salonen R, Kauhanen J, Valkonen V-P, Pesonen U, Koulu M, Scheinin M, Salonen J T. An Insertion/Deletion polymorphism in the α₂₈-adrenergic receptor gene is a novel genetic risk factor for acute coronary events. J Am Coll Cardiol 2001; 37:1516-1522.

Syvänen A-C: Accessing genetic variation: genotyping single nucleotide polymorphisms. Nature reviews/Genetics 2:930-942, 2001

Tuomainen T-P, Kontula K, Nyyssönen K, Lakka T A, Heliö T, Salonen J T. Increased risk of acute myocardial infarction in carriers of the hemochromatosis gene Cys282Tyr mutation: A prospective cohort study in men in Eastern Finland. Circulation 1999; 100: 1274-1279.

Valore E V, Park C H, Quayle A J, Wiles K R, McCray P B Jr, Ganz T. Human beta-defensin-1: an antimicrobial peptide of urogenital tissues J Clin Invest 1998; 101: 1633-42.

1. A method for detecting genetic variation or polymorphism, i.e. a mutation, in a defensin gene comprising the steps of:
i) providing a biological sample taken from a subject to be tested,
ii) detecting the presence or absence of a variant genotype of the defensin gene in the biological sample, the presence of a variant defensin genotype indicating an increased risk of cardiovascular disease in said subject.

2. The method according to claim 1, wherein said variant genotype of the defensin gene is a homo- or heterozygote form of the mutation.

3. The method according to claim 1, wherein the detection step is a DNA-assay.

4. The method according to claim 1, wherein the detection step is carried out using a gene or DNA chip, microarray, strip, panel or similar combination of more than one genes, mutations or RNA expressions to be assayed.

5. The method according to claim 1, wherein the allelic pattern is determined using polymerase chain reaction.

6. The method according to claim 1, wherein the biological sample is a blood sample or buccal swab sample and genomic DNA is isolated from said sample.

7. The method according to claim 1, wherein the detection step is based on a capturing probe.

8. The method according to claim 1, wherein said method is used for determining whether a subject will benefit from treatment with a drug, nutrient or other therapy enhancing the defensin production, levels or activity or inhibiting defensin catabolism or elimination in the subject.

9. The method according to claim 1, wherein said method is used for determining whether a subject will be at increased risk of adverse effects or reactions if defensin antagonists are administered to a subject.

10. The method according to claim 1, further comprising a step of selecting a subject with a defensin gene sequence reducing the expression, production or levels of defensin protein for clinical drug trials testing the anticoronary and myocardial ischaemia preventing effects of compounds.

11. The method according to claim 1, wherein said cardiovascular disease is acute myocardial infarction (AMI) or coronary heart disease (CHD).

12. The method according to claim 1, wherein said defensin is selected from the group consisting of: beta-defensin-1, beta-defensin-129, and alfa-defensin-5.

13. The method according to claim 12, wherein said variant genotype is human beta-defensin-1 gene comprising 3′UTR+5A→G mutation.

14. The method according to claim 12, wherein said variant genotype is human alfa-defensin-5 gene comprising IVS1+198C→T mutation and/or IVS1+243G→C mutation.

15. The method according to claim 12, wherein said variant genotype is human beta-defensin- 129 gene comprising IVS1-13_{—}12insCTC mutation.

16. The method according to claim 1, wherein genetic variation is further determined from the genes selected from the group consisting of:
a) alpha-_{2B}-adrenoceptor,
b) apolipoprotein B, and
c) beta-2-adrenergic receptor
wherein the presence of a variant genotype in said genes indicates an increased risk of cardiovascular disease in said subject.

17. The method according to claim 16, wherein said variant genotype is alpha-_{2B}-adrenoceptor gene comprising insertion/deletion mutation, or said variant genotype is beta-2-adrenergic receptor comprising Gly16Arg and/or Glu27Gln mutation.

18. The method according to claim 16, wherein said variant genotype is apolipoprotein B gene comprising Thr98Ile mutation.

19. The method according to claim 1, further comprising a step of combining information concerning age, gender, the family history of hypertension, diabetes and hypercholesterolemia, and the medical history concerning cardiovascular diseases or diabetes of the subject with the results obtained from step ii) of the method for confirming the indication obtained from the detection step.

20. The method according to claim 19, wherein said information is about hypercholesterolemia in the family, smoking status, use of cholesterol lowering medications, CHD in the family, history of cardiovascular disease, obesity in the family, and waist-to-hip circumference ratio (cm/cm).

21. The method according to claim 1, further comprising a step determining blood, serum or plasma cholesterol, HDL cholesterol, LDL cholesterol, triglyceride, apolipoprotein B and AI, fibrinogen, ferritin, transferrin receptor, C-reactive protein, serum or plasma insulin concentration.

22. The method according to claims 12, wherein the detected mutations are 3′UTR+5 A/G of the beta-defensin-1 gene, an insertion/deletion of three glutamic acids in the region of 12 Glu aminoacids in the codons 298-309 of Alpha-_{2B}-adrenoceptor, and the Thr98Ile of apolipoprotein B gene.

23. The method according to claim 1 further comprising a step of calculating the probability of a cardiovascular disease using a logistic regression equation as follows:
probability of a cardiovascular disease=[1+e^{(-(-a+Σ(bi*Xi))}]⁻¹
where e is Napier's constant, Xᵢ are variables related to the cardiovascular disease, bᵢ are coefficients of these variables in the logistic function, and a is the constant term in the logistic function.

24. The method according to claim 23, wherein a and bi are determined in the population in which the method is to be used.

25. The method according to claim 23, wherein Xi are selected among the variables that have been measured in the population in which the method is to be used.

26. The method according to claim 23, wherein bᵢ are between the values of −20 and 20 and/or wherein Xᵢ are binary variables that can have values or are coded as 0 (zero) or 1 (one).

27. The method according to claim 23, wherein i are between the values 0 (none) and 100,000.

28. The method according to claim 23, wherein subject's short term, median term, and/or long term risk of CHD and/or AMI is predicted.

29. A method for targeting the treatment of CHD and AMI in a subject with CHD by determining the pattern of alleles encoding a variant defensin, i.e. by determining if said subject's genotype of the defensin is of the variant type, comprising the steps presented in claim 1, and treating a subject of the variant genotype with a drug affecting defensin production or metabolism of the subject.

30. The method according to claim 29, wherein said defensin is as defined in claim 12.

31. The method according to claim 30, wherein the variant genotype is as defined in any one of claims 13-15.

32. The method according to claim any one of claim 29, wherein said variant genotype of the defensin is a homozygote or heterozygote form of mutation.

33. The method according to claim 29, wherein said CHD is angina pectoris or other form of CHD.

34. A method for treating a human or animal suffering from CHD or AMI, said method comprising;
providing said human or animal with a therapy, wherein said therapy enhances defensin availability, enhances defensin production or enhances defensin concentration in the human subject or animal.

35. The method of claim 34, wherein said animal is a mammal.

36. A method for treating vascular complications of CHD and AMI, said method comprising a step of enhancing defensin availability, production or concentration in the circulation of a human subject or animal.

37. The method according to claim 34 or 36, wherein said defensin is as defined in claim 12.

38. The method according to claim 34 or 36, wherein said defensin is Beta-defensin-1.

39. The method according to claim 34 or 36, wherein the said method of treating is selected from at least one of the group consisting of: a dietary treatment, a vaccination, gene therapy and gene transfer.

40. (canceled)

41. The method according to claim 39, wherein said therapy comprises the transfer of the non-variant Beta-defensin 1 gene or fragment or derivative thereof.

42. A kit for detecting genetic variation or polymorphism, i.e. a mutation, in a defensin gene for the determination of a risk of acute myocardial infarction, AMI, and coronary heart disease, CHD, in a subject, comprising means for defensin gene allele detection, and optionally software to interpret the results of the determination.

43. The kit according to claim 42, wherein said defensin is as defined in claim 12.

44. The kit according to claim 42, wherein genetic variation or polymorphism, i.e. a mutation, is further detected in the genes selected from the group consisting of:
a) alpha_{2B}-adrenoceptor
b) apolipoprotein B, and
c) beta-2-adrenergic receptor.

45. The method according to claim 44, wherein the genetic variation to be detected is as defined in any one of claims 13-15.

46. The kit according to claim 42 comprising a capturing nucleic acid probe specifically binding to the variant genotype as defined in any one of claims 13-15.

47. The kit according to claim 42, comprising a DNA chip, microarray, DNA strip, DNA panel or real-time PCR based tests.

48. The kit according to claim 42, comprising a questionnaire for obtaining patient information concerning age, gender, height, weight, the family history of hypertension and hypercholesterolemia, the medical history concerning cardiovascular diseases.

49. An isolated variant nucleic acid encoding alfa-defensin-5 protein, said nucleic acid comprising IVS1+198C→T and/or IVS1+243G→C mutation.

50. An isolated variant nucleic acid encoding beta-defensin-129 protein, said nucleic acid comprising IVS1-13_{—}12 in/del CTC mutation.

51. The nucleic acid according to claim 49 or 50, wherein said nucleic acid is a genomic nucleotide sequence.

52. The nucleic acid according to claim 51, wherein said nucleic acid is cDNA.

53. The nucleic acid according to claim 49 or 50 comprising an RNA sequence.

54. The nucleic acid according to claim 49 having the nucleic acid sequence set forth in SEQ ID NO:7.

55. The nucleic acid according to claim 50 having the nucleic acid sequence set forth in SEQ ID NO:32.

56. A capturing probe binding to the nucleic acid according to claim 49 or 50.

57. A capturing probe binding to the nucleic acid according to claim 49 or 50, wherein said probe comprises a single strand of genomic nucleotide sequence and said genomic nucleotide sequence is cDNA.

58. The capturing probe according to claim 56, which specifically binds to a variant defensin nucleic acid encoding
alfa-defensin-5 protein, said nucleic acid comprising IVS1+198C→T and/or IVS1+243G→C mutation, or
beta-defensin-129 protein, said nucleic acid comprising IVS1-13_{—}12 in/del CTC mutation.
wherein said capturing probe does not bind non-variant defensin.

59. A method for determining the presence or absence of a nucleic acid as defined in claim 49 or 50 in a biological sample comprising the steps of:
a) treating said sample to obtain single stranded target nucleic acid, or if the target nucleic acid are already single stranded, directly employing step (b);
b) contacting said target nucleic acid with a capturing nucleic acid probe and a detector nucleic acid probe;
c) detecting the complex of capturing probe, target nucleic acid and detector probe.

60. The method according to claim 59, wherein the capturing nucleic acid probe is attached or capable of attaching to a solid phase, and wherein said probe comprises a single strand of genomic nucleotide sequence and said genomic nucleotide sequence is cDNA, and wherein a detected signal from the solid phase is an indication of the presence in the sample of a nucleic acid encoding
alfa-defensin-5 protein, said nucleic acid comprising IVS1+198C→T and/or IVS1+243G→C mutation, or
beta-defensin-129 protein, said nucleic acid comprising IVS1-13_{—}12 in/del CTC mutation.

61. The method according to claim 60, wherein the capturing nucleic acid probe is attached or capable of attaching to a solid phase, and comprises a cDNA corresponding to the gene coding a wild-type defensin protein.

62. A transgenic animal which carries a human DNA sequence comprising a nucleotide sequence encoding a variant defensin nucleic acid as defined in claim 49 or 50.

63. RNA interference methods and models involving a variant nucleotide sequence encoding a variant defensin nucleic acid as defined in claim 49 or 50.

64. A method for measuring defensin protein expression, production or concentration in human tissues, comprising the steps of:
a) providing a tissue sample taken from a subject to be tested,
b) detecting the expression, production or concentration of defensin protein in said sample, wherein reduced expression, production or concentration indicates an increased risk of cardiovascular disease in said subject.


SEQUENCE LISTING (56 sequences)

SEQ ID NO: 1 - DNA, 20 bp, Artificial Sequence
  Description of Artificial Sequence PCR primer
cataatttcagcccgatgtg

SEQ ID NO: 2 - DNA, 20 bp, Artificial Sequence
  Description of Artificial Sequence PCR primer
caccctaaccccctacttct

SEQ ID NO: 3 - DNA, 18 bp, Artificial Sequence
  Description of Artificial Sequence PCR primer
gggcttgctctttctttc

SEQ ID NO: 4 - DNA, 18 bp, Artificial Sequence
  Description of Artificial Sequence PCR primer
tccttggttcctctcatc

SEQ ID NO: 5 - DNA, 18 bp, Artificial Sequence
  Description of Artificial Sequence PCR primer
ctgagtgtgcaggacgag

SEQ ID NO: 6 - DNA, 18 bp, Artificial Sequence
  Description of Artificial Sequence PCR primer
cacattgccaaacacgat

SEQ ID NO: 7 - DNA, 736 bp, Homo sapiens
agaaagaggagcatcaaagggatcttgagaacaaaggcagtccttcccctcccaatcacatgcccacctcctctcactgcagcttctgtctcaggtcttctcccagcagagctataaatccaggctgactcctcactccccacatatccactcctgctctccctcctgcaggtgaccccagccatgaggaccatcgccatccttgctgccattctcctggtggccctgcaggcccaggctgagtcactccaggaaagagctgatgaggctacaacccagaagcagtctggggaagacaaccaggaccttgctatctcctttgcaggaaatggactctctgctcttagaacctcaggtaggagacatcaatcttgcacatctgcaaaatctagaaaaaaaggattggagaaaggatctggagtcaagtgtggaaaggtctacctcacttgagtgactttacttaatcttcctggaccttgattttctcatctataaattaatcagtgagaaccaaataaatctaaaagattttcttttttctaagactttcagttccaagatatttctgtgaaatttgctacttttaagatagaaagacctacactgactagttctttgtagatctaaatgggcagacttagttatatagagagtgttttactttgtccattggaaaagcttttagaacctagagaggaacctataggtgtgttttgatgtaggctaataggcttga

SEQ ID NO: 8 - DNA, 736 bp, Homo sapiens
agaaagaggagcatcaaagggatcttgagaacaaaggcagtccttcccctcccaatcacatgcccacctcctctcactgcagcttctgtctcaggtcttctcccagcagagctataaatccaggctgactcctcactccccacatatccactcctgctctccctcctgcaggtgaccccagccatgaggaccatcgccatccttgctgccattctcctggtggccctgcaggcccaggctgagtcactccaggaaagagctgatgaggctacaacccagaagcagtctggggaagacaaccaggaccttgctatctcctttgcaggaaatggactctctgctcttagaacctcaggtaggagacatcaatcttgcacatctgcaaaatctagaaaaaaaggattggagaaaggatctggagtcaagtgtggaaaggtctacctcacttgagtgactttacttaatcttcctggaccttgattttctcatctataaattaatcagtgagaaccaaataaatctaaaagattttcttttttctaagactttcagctccaagatatttctgtgaaatttgctacttttaagatagaaagagctacactgactagttctttgtagatctaaatgggcagacttagttatatagagagtgttttactttgtccattggaaaagcttttagaacctagagaggaacctataggtgtgttttgatgtaggctaataggcttga

SEQ ID NO: 9 - DNA, 19 bp, Artificial Sequence
  Description of Artificial Sequence PCR primer
agaaagaggagcatcaaag

SEQ ID NO: 10 - DNA, 19 bp, Artificial Sequence
  Description of Artificial Sequence PCR primer
tcaagcctattagcctaca

SEQ ID NO: 11 - DNA, 20 bp, Artificial Sequence
  Description of Artificial Sequence PCR primer
gacaacctcaatgctctgct

SEQ ID NO: 12 - DNA, 20 bp, Artificial Sequence
  Description of Artificial Sequence PCR primer
tgacttacctggacatggct

SEQ ID NO: 13 - DNA, 35 bp, Artificial Sequence
  Snapshot primer
tttttttttttttttcttttttctaagactttcag

SEQ ID NO: 14 - DNA, 40 bp, Artificial Sequence
  Snapshot primer
tttttttttttttttttttgctacttttaagatagaaaga

SEQ ID NO: 15 - DNA, 45 bp, Artificial Sequence
  Snapshot primer
tttttttttttttttttttttttttttagtgctgcaagtgagctg

SEQ ID NO: 16 - DNA, 50 bp, Artificial Sequence
  Snapshot primer
tttttttttttttttttttttttttttttttttccagagaggaagccttg

SEQ ID NO: 17 - DNA, 55 bp, Artificial Sequence
  Snapshot primer
tttttttttttttttttttttttttttttttttttttttcttgctggcacccaat

SEQ ID NO: 18 - DNA, 60 bp, Artificial Sequence
  Snapshot primer
tttttttttttttttttttttttttttttttttttttttttttaccacgacgtcacgcag

SEQ ID NO: 19 - DNA, 30 bp, Artificial Sequence
  Snapshot primer
tttttttttttttgaagaccagccagtgca

SEQ ID NO: 20 - DNA, 1344 bp, Homo sapiens
  CDS (1)..(1344): Coding sequence for variant human ADRA2B gene
atggaccaccaggacccctactccgtgcaggccacagcggccatagcggcggccatcaccttcctcattctctttaccatcttcggcaacgctctggtcatcctggctgtgttgaccagccgctcgctgcgcgcccctcagaacctgttcctggtgtcgctggccgccgccgacatcctggtggccacgctcatcatccctttctcgctggccaacgagctgctgggctactggtacttccggcgcacgtggtgcgaggtgtacctggcgctcgacgtgctcttctgcacctcgtccatcgtgcacctgtgcgccatcagcctggaccgctactgggccgtgagccgcgcgctggagtacaactccaagcgcaccccgcgccgcatcaagtgcatcatcctcactgtgtggctcatcgccgccgtcatctcgctgccgcccctcatctacaagggcgaccagggcccccagccgcgcgggcgcccccagtgcaagctcaaccaggaggcctggtacatcctggcctccagcatcggatctttctttgctccttgcctcatcatgatccttgtctacctgcgcatctacctgatcgccaaacgcagcaaccgcagaggtcccagggccaagggggggcctgggcagggtgagtccaagcagccccgacccgaccatggtggggctttggcctcagccaaactgccagccctggcctctgtggcttctgccagagaggtcaacggacactcgaagtccactggggagaaggaggagggggagacccctgaagatactgggacccgggccttgccacccagttgggctgcccttcccaactcaggccagggccagaaggagggtgtttgtggggcatctccagaggatgaagctgaagaggaggaagaggaggaggaggagtgtgaaccccaggcagtgccagtgtctccggcctcagcttgcagccccccgctgcagcagccacagggctcccgggtgctggccaccctacgtggccaggtgctcctgggcaggggcgtgggtgctataggtgggcagtggtggcgtcgaagggcgcacgtgacccgggagaagcgcttcaccttcgtgctggctgtggtcattggcgtttttgtgctctgctggttccccttcttcttcagctacagcctgggcgccatctgcccgaagcactgcaaggtgccccatggcctcttccagttcttcttctggatcggctactgcaacagctcactgaaccctgttatctacaccatcttcaaccaggacttccgccgtgccttccggaggatcctgtgccgcccgtggacccagacggcctggtga

SEQ ID NO: 21 - protein, 447 aa, Homo sapiens
MDHQDPYSVQATAAIAAAITFLILFTIFGNALVILAVLTSRSLRAPQNLFLVSLAAADILVATLIIPFSLANELLGYWYFRRTWCEVYLALDVLFCTSSIVHLCAISLDRYWAVSRALEYNSKRTPRRIKCIILTVWLIAAVISLPPLIYKGDQGPQPRGRPQCKLNQEAWYILASSIGSFFAPCLIMILVYLRIYLIAKRSNRRGPRAKGGPGQGESKQPRPDHGGALASAKLPALASVASAREVNGHSKSTGEKEEGETPEDTGTRALPPSWAALPNSGQGQKEGVCGASPEDEAEEEEEEEEECEPQAVPVSPASACSPPLQQPQGSRVLATLRGQVLLGRGVGAIGGQWWRRRAHVTREKRFTFVLAVVIGVFVLCWFPFFFSYSLGAICPKHCKVPHGLFQFFFWIGYCNSSLNPVIYTIFNQDFRRAFRRILCRPWTQTAW

SEQ ID NO: 22 - DNA, 1353 bp, Homo sapiens
  CDS (1)..(1353): Coding sequence for human ADRA2B gene
atggaccaccaggacccctactccgtgcaggccacagcggccatagcggcggccatcaccttcctcattctctttaccatcttcggcaacgctctggtcatcctggctgtgttgaccagccgctcgctgcgcgcccctcagaacctgttcctggtgtcgctggccgccgccgacatcctggtggccacgctcatcatccctttctcgctggccaacgagctgctgggctactggtacttccggcgcacgtggtgcgaggtgtacctggcgctcgacgtgctcttctgcacctcgtccatcgtgcacctgtgcgccatcagcctggaccgctactgggccgtgagccgcgcgctggagtacaactccaagcgcaccccgcgccgcatcaagtgcatcatcctcactgtgtggctcatcgccgccgtcatctcgctgccgcccctcatctacaagggcgaccagggcccccagccgcgcgggcgcccccagtgcaagctcaaccaggaggcctggtacatcctggcctccagcatcggatctttctttgctccttgcctcatcatgatccttgtctacctgcgcatctacctgatcgccaaacgcagcaaccgcagaggtcccagggccaagggggggcctgggcagggtgagtccaagcagccccgacccgaccatggtggggctttggcctcagccaaactgccagccctggcctctgtggcttctgccagagaggtcaacggacactcgaagtccactggggagaaggaggagggggagacccctgaagatactgggacccgggccttgccacccagttgggctgcccttcccaactcaggccagggccagaaggagggtgtttgtggggcatctccagaggatgaagctgaagaggaggaagaggaggaggaggaggaggaagagtgtgaaccccaggcagtgccagtgtctccggcctcagcttgcagccccccgctgcagcagccacagggctcccgggtgctggccaccctacgtggccaggtgctcctgggcaggggcgtgggtgctataggtgggcagtggtggcgtcgaagggcgcacgtgacccgggagaagcgcttcaccttcgtgctggctgtggtcattggcgtttttgtgctctgctggttccccttcttcttcagctacagcctgggcgccatctgcccgaagcactgcaaggtgccccatggcctcttccagttcttcttctggatcggctactgcaacagctcactgaaccctgttatctacaccatcttcaaccaggacttccgccgtgccttccggaggatcctgtgccgcccgtggacccagacggcctggtga

SEQ ID NO: 23 - protein, 450 aa, Homo sapiens
MDHQDPYSVQATAAIAAAITFLILFTIFGNALVILAVLTSRSLRAPQNLFLVSLAAADILVATLIIPFSLANELLGYWYFRRTWCEVYLALDVLFCTSSIVHLCAISLDRYWAVSRALEYNSKRTPRRIKCIILTVWLIAAVISLPPLIYKGDQGPQPRGRPQCKLNQEAWYILASSIGSFFAPCLIMILVYLRIYLIAKRSNRRGPRAKGGPGQGESKQPRPDHGGALASAKLPALASVASAREVNGHSKSTGEKEEGETPEDTGTRALPPSWAALPNSGQGQKEGVCGASPEDEAEEEEEEEEEEEECEPQAVPVSPASACSPPLQQPQGSRVLATLRGQVLLGRGVGAIGGQWWRRRAHVTREKRFTFVLAVVIGVFVLCWFPFFFSYSLGAICPKHCKVPHGLFQFFFWIGYCNSSLNPVIYTIFNQDFRRAFRRILCRPWTQTAW

SEQ ID NO: 24 - DNA, 20 bp, Artificial Sequence
  Description of Artificial Sequence PCR primer
gggtgtttgtggggcatctc

SEQ ID NO: 25 - DNA, 19 bp, Artificial Sequence
  Snapshot primer
tggcactgcctggggttca

SEQ ID NO: 26 - DNA, 18 bp, Artificial Sequence
  Sequencing primer
tcaggtcttctcccagca

SEQ ID NO: 27 - DNA, 619 bp, Homo sapiens
ggatgaagcagaatgaagagtaggtaaccctgaggttgagaggtatattgttggaccagggagcaggtaataaatacatcctggatagactcacatggggaaaaaaactatgatcttgcatgactaacacatagctagtaagatttcttgtcacttacgacaaagacatgaattttctccatcctaacatgactgatacagtgtctcttatttagactatctcagttagtctggctgtgcttgtcctttttcccacctccctcgctgtgcctgaccctctcttctttccacaggttctcaggcaagagccacctgctattgccgaaccggccgttgtgctacccgtgagtccctctccggggtgtgtgaaatcagtggccgcctctacagactctgctgtcgctgagcttcctagatagaaaccaaagcagtgcaagattcagttcaaggtcctgaaaaaagaaaaacattttactctgtgtaccttgtgtctttctaaatttctctctccaaagtaaagttcaagcattaaacttagtgtgtttgacctttttaattttcttttctttttccttttttttcttttgctttgttatatggtggtttgtatggttccttt

SEQ ID NO: 28 - DNA, 619 bp, Homo sapiens
ggatgaagcagaatgaagagtaggtaaccctgaggttgagaggtatattgttggaccagggagcaggtaataaatacatcctggatagactcacatggggaaaaaaactatgatcttgcatgactaacacatagctagtaagatttcttgtcacttacgacaaagacatgaattttctccatcctaacatgactgatacagtgtctcttatttagactatctcagttagtctggctgtgcttgtcctttttcccacctccctcgctgtgcctgaccctctcttctttccacaggttctcaggcaagagccacctgctattgccgaaccggccgttgtgctacccgtgagtccctctccggggtgtgtgaaatcagtggccgcctctacagactctgctgtcgctgagcttcctagatagaaaccaaagcagtgcaagattcagttcaaggtcctgaaaaaagaaaaacattttactctgtgtaccttgtgtctttctaaatttctctctccaaaataaagttcaagcattaaacttagtgtgtttgacctttttaattttcttttctttttccctttttttcttttgctttgttatatggtggtttgtatggttccttt

SEQ ID NO: 29 - DNA, 19 bp, Artificial Sequence
  Description of Artificial Sequence PCR primer
ggatgaagcagaatgaaga

SEQ ID NO: 30 - DNA, 19 bp, Artificial Sequence
  Description of Artificial Sequence PCR primer
aaaggaaccatacaaacca

SEQ ID NO: 31 - DNA, 18 bp, Artificial Sequence
  Sequencing primer
gttagtctggctgtgctt

SEQ ID NO: 32 - DNA, 1052 bp, Homo sapiens
gggctactgagtttggtgaaaagataagactcctgaaaattctattgattctcttttgaacttctttcttaaattagttttatgatggacttggctctcattggtatttcccaagattatggagatgggatagtgatgtctgacaagtacctaagatgctaagttgaaggtctaaaattccatcctaaaagcaaataattactctatcatctacgtgccctttgcttcttaaagttactcaaggaaggcagactaaacaggaaatttactttggattcaagaggggcatagagacgctctcagcctgcccatttgccttcatcaacattcctaaacactgggcttaaaatgtagtatgagtaaactctctcttagtctatccatctcccactagcagttttaacatcatctctagttattaaccttggctcaatggctttctcctctttttttatacagaatttattggcttgagacgctgtttaatgggtttggggagatgcagggatcactgcaatgtggatgaaaaagagatacagaaatgcaagatgaaaaaatgttgtgttggaccaaaagtggttaaattgattaaaaactacctgcaatatggaacaccaaatgtacttaatgaagacgtccaagaaatgctaaaacctgccaagaattctagtgctgtgatacaaagaaaacatattttatctgttctcccccaaatcaaaagcactagcttttttgctaataccaactttgtcatcattccaaatgccacccctatgaactctgccaccatcagcactatgaccccaggacagatcacatacactgctacttctaccaagagtaacaccaaagaaagcagagattctgccactgcctcgccaccaccagcaccacctccaccaaacatactgccaacaccatcactggagctagaggaagcagaagagcagtaatgtggatctttcccttaaaactccaagttcctctctatttttgctatctataaaatgacatagaactgtttcctctgtcatcagtcattcaataaacac

SEQ ID NO: 33 - DNA, 1049 bp, Homo sapiens
gggctactgagtttggtgaaaagataagactcctgaaaattctattgattctcttttgaacttctttcttaaattagttttatgatggacttggctctcattggtatttcccaagattatggagatgggatagtgatgtctgacaagtacctaagatgctaagttgaaggtctaaaattccatcctaaaagcaaataattactctatcatctacgtgccctttgcttcttaaagttactcaaggaaggcagactaaacaggaaatttactttggattcaagaggggcatagagacgctctcagcctgcccatttgccttcatcaacattcctaaacactgggcttaaaatgtagtatgagtaaactctctcttagtctatccatctcccactagcagttttaacatcatctctagttattaaccttggctcaatggctttctctttttttatacagaatttattggcttgagacgctgtttaatgggtttggggagatgcagggatcactgcaatgtggatgaaaaagagatacagaaatgcaagatgaaaaaatgttgtgttggaccaaaagtggttaaattgattaaaaactacctgcaatatggaacaccaaatgtacttaatgaagacgtccaagaaatgctaaaacctgccaagaattctagtgctgtgatacaaagaaaacatattttatctgttctcccccaaatcaaaagcactagcttttttgctaataccaactttgtcatcattccaaatgccacccctatgaactctgccaccatcagcactatgaccccaggacagatcacatacactgctacttctaccaagagtaacaccaaagaaagcagagattctgccactgcctcgccaccaccagcaccacctccaccaaacatactgccaacaccatcactggagctagaggaagcagaagagcagtaatgtggatctttcccttaaaactccaagttcctctctatttttgctatctataaaatgacatagaactgtttcctctgtcatcagtcattcaataaacac

SEQ ID NO: 34 - DNA, 18 bp, Artificial Sequence
  Description of Artificial Sequence PCR primer
ggctactgagtttggtga

SEQ ID NO: 35 - DNA, 21 bp, Artificial Sequence
  Description of Artificial Sequence PCR primer
gtgtttattgaatgactgatg

SEQ ID NO: 36 - DNA, 18 bp, Artificial Sequence
  Sequencing primer
caaggaaggcagactaaa

SEQ ID NO: 37 - DNA, 552 bp, Homo sapiens
  CDS (1)..(552): Coding sequence for the variant human DEFB129 gene
atgaagctcctttttcctatctttgccagcctcatgctacagtaccaggtgaacacagaatttattggcttgagacgctgtttaatgggtttggggagatgcagggatcactgcaatgtggatgaaaaagagatacagaaatgcaagatgaaaaaatgttgtgttggaccaaaagtggttaaattgattaaaaactacctgcaatatggaacaccaaatgtacttaatgaagacgtccaagaaatgctaaaacctgccaagaattctagtgctgtgatacaaagaaaacatattttatctgttctcccccaaatcaaaagcactagcttttttgctaataccaactttgtcatcattccaaatgccacccctatgaactctgccaccatcagcactatgaccccaggacagatcacatacactgctacttctaccaagagtaacaccaaagaaagcagagattctgccactgcctcgccaccaccagcaccacctccaccaaacatactgccaacaccatcactggagctagaggaagcagaagagcagtaa

SEQ ID NO: 38 - protein, 183 aa, Homo sapiens
MKLLFPIFASLMLQYQVNTEFIGLRRCLMGLGRCRDHCNVDEKEIQKCKMKKCCVGPKVVKLIKNYLQYGTPNVLNEDVQEMLKPAKNSSAVIQRKHILSVLPQIKSTSFFANTNFVIIPNATPMNSATISTMTPGQITYTATSTKSNTKESRDSATASPPPAPPPPNILPTPSLELEEAEEQ

SEQ ID NO: 39 - DNA, 552 bp, Homo sapiens
  CDS (1)..(552): Coding sequence for the human DEFB129 gene
atgaagctcctttttcctatctttgccagcctcatgctacagtaccaggtgaacacagaatttattggcttgagacgctgtttaatgggtttggggagatgcagggatcactgcaatgtggatgaaaaagagatacagaaatgcaagatgaaaaaatgttgtgttggaccaaaagtggttaaattgattaaaaactacctacaatatggaacaccaaatgtacttaatgaagacgtccaagaaatgctaaaacctgccaagaattctagtgctgtgatacaaagaaaacatattttatctgttctcccccaaatcaaaagcactagcttttttgctaataccaactttgtcatcattccaaatgccacccctatgaactctgccaccatcagcactatgaccccaggacagatcacatacactgctacttctaccaagagtaacaccaaagaaagcagagattctgccactgcctcgccaccaccagcaccacctccaccaaacatactgccaacaccatcactggagctagaggaagcagaagagcagtaa

SEQ ID NO: 40 - protein, 183 aa, Homo sapiens; identical to SEQ ID NO: 38

SEQ ID NO: 41 - DNA, 372 bp, Homo sapiens
  CDS (1)..(372): Coding sequence for the variant human DEFB118 gene
atgaaactcctgctgctggctcttcctatgcttgtgctcctaccccaagtgatcccagcctatagtggtgaaaaaaaatgctggaacagatcagggcaccgcaggaaacaatgcaaagatggagaagcagtgaaagatacatgcaaaaatcttcgagcttgctgcattccatccaatgaagaccacaggcgagttcctgcgacatctcccacacccttgagtgactcaacaccaggaattattgatgatattttaacagtaaggttcacgacagactactttgaagtaagcagcaagaaagatatggttgaagagtctgaggcgggaaggggaactgagacctctcttccaaatgttcaccatagctcatga

SEQ ID NO: 42 - protein, 123 aa, Homo sapiens
MKLLLLALPMLVLLPQVIPAYSGEKKCWNRSGHRRKQCKDGEAVKDTCKNLRACCIPSNEDHRRVPATSPTPLSDSTPGIIDDILTVRFTTDYFEVSSKKDMVEESEAGRGTETSLPNVHHSS

SEQ ID NO: 43 - DNA, 372 bp, Homo sapiens
  CDS (1)..(372): Coding sequence of the human DEFB118 gene
atgaaactcctgctgctggctcttcctatgcttgtgctcctaccccaagtgatcccagcctatagtggtgaaaaaaaatgctggaacagatcagggcactgcaggaaacaatgcaaagatggagaagcagtgaaagatacatgcaaaaatcttcgagcttgctgcattccatccaatgaagaccacaggcgagttcctgcgacatctcccacacccttgagtgactcaacaccaggaattattgatgatattttaacagtaaggttcacgacagactactttgaagtaagcagcaagaaagatatggttgaagagtctgaggcgggaaggggaactgagacctctcttccaaatgttcaccatagctcatga

SEQ ID NO: 44 - protein, 123 aa, Homo sapiens
MKLLLLALPMLVLLPQVIPAYSGEKKCWNRSGHCRKQCKDGEAVKDTCKNLRACCIPSNEDHRRVPATSPTPLSDSTPGIIDDILTVRFTTDYFEVSSKKDMVEESEAGRGTETSLPNVHHSS

SEQ ID NO: 45 - DNA, 20 bp, Artificial Sequence
  Description of Artificial Sequence PCR primer
aggttgagtatttgccagac

SEQ ID NO: 46 - DNA, 19 bp, Artificial Sequence
  Description of Artificial Sequence PCR primer
aggacaggggtgagtgata

SEQ ID NO: 47 - DNA, 246 bp, Homo sapiens
  CDS (1)..(246): Coding sequence for the variant human DEFB126 gene
atgaagtccctactgttcacccttgcagtttttatgctcctggcccaattggtctcaggtaattggtatgtgaaaaagtgtctaaacgacgttggaatttgcaagaagaagtgcaaacctgaagagatgcatgtaaagaatggttgggcaatgtgcggcaaagggactgctgtgttccagctgacagacgtgctaattatcctgttttctgtgtccagacaaagactacaagaatttcaacagtaa

SEQ ID NO: 48 - protein, 81 aa, Homo sapiens
MKSLLFTLAVFMLLAQLVSGNWYVKKCLNDVGICKKKCKPEEMHVKNGWAMCGKGTAVFQLTDVLIILFSVSRQRLQEFQQ

SEQ ID NO: 49 - DNA, 336 bp, Homo sapiens
  CDS (1)..(336): Coding sequence of the human DEFB126 gene
atgaagtccctactgttcacccttgcagtttttatgctcctggcccaattggtctcaggtaattggtatgtgaaaaagtgtctaaacgacgttggaatttgcaagaagaagtgcaaacctgaagagatgcatgtaaagaatggttgggcaatgtgcggcaaacaaagggactgctgtgttccagctgacagacgtgctaattatcctgttttctgtgtccagacaaagactacaagaatttcaacagtaacagcaacaacagcaacaacaactttgatgatgactactgcttcgatgtcttcgatggctcctacccccgtttctcccactggttga

SEQ ID NO: 50 - protein, 111 aa, Homo sapiens
MKSLLFTLAVFMLLAQLVSGNWYVKKCLNDVGICKKKCKPEEMHVKNGWAMCGKQRDCCVPADRRANYPVFCVQTKTTRISTVTATTATTTLMMTTASMSSMAPTPVSPTG

SEQ ID NO: 51 - DNA, 20 bp, Artificial Sequence
  Description of Artificial Sequence PCR primer
aatggtgagaaagatgacag

SEQ ID NO: 52 - DNA, 18 bp, Artificial Sequence
  Description of Artificial Sequence PCR primer
gttgaatggagggaaagt

SEQ ID NO: 53 - DNA, 18 bp, Artificial Sequence
  Sequencing primer
gtaggtatttatgattag

SEQ ID NO: 54 - DNA, 334 bp, Homo sapiens
  CDS (1)..(333): Coding sequence for the variant human DEFB126 gene
atgaagtccctactgttcacccttgcagtttttatgctcctggcccaattggtctcaggtaattggtatgtgaaaaagtgtctaaacgacgttggaatttgcaagaagaagtgcaaacctgaagagatgcatgtaaagaatggttgggcaatgtgcggcaaacaaagggactgctgtgttccagctgacagacgtgctaattatcctgttttctgtgtccagacaaagactacaagaatttcaacagtaacagcaacaacagcaacaacaactttgatgatgactactgcttcgatgtcttcgatggctcctacccgtttctcccactggttga

SEQ ID NO: 55 - protein, 111 aa, Homo sapiens
MKSLLFTLAVFMLLAQLVSGNWYVKKCLNDVGICKKKCKPEEMHVKNGWAMCGKQRDCCVPADRRANYPVFCVQTKTTRISTVTATTATTTLMMTTASMSSMAPTRFSHWL

SEQ ID NO: 56 - DNA, 50 bp, Artificial Sequence
  Description of Artificial Sequence Synthetic snapshot primer
tttttttttttttttttttttttttttttttttgctcaatggctttctct